(19) United States
(12) Patent Application Publication
(10) Pub. No.: US 2020/0297308 A1
(43) Pub. Date: Sep. 24, 2020

(54) ULTRASOUND AUTOMATIC SCANNING SYSTEM, ULTRASOUND DIAGNOSIS APPARATUS, AND ULTRASOUND SCANNING SUPPORT APPARATUS

(71) Applicant: CANON MEDICAL SYSTEMS CORPORATION, Otawara-shi (JP)

(72) Inventors: Yusuke KOBAYASHI, Nasushiobara (JP); Kengo OKADA, Nasushiobara (JP)

(73) Assignee: CANON MEDICAL SYSTEMS CORPORATION, Otawara-shi (JP)

(21) Appl. No.: 16/823,509

(22) Filed: Mar. 19, 2020

(30) Foreign Application Priority Data
Mar. 19, 2019 (JP) 2019-050609

(51) Int. Cl. A61B 8/00

(52) U.S. Cl. CPC A61B 8/4245; A61B 34/37; A61B 8/4466; A61B 8/4218

(57) ABSTRACT

In an ultrasound automatic scanning system according to an embodiment, one or more ultrasound probes transmit and receive an ultrasound wave. A mechanical mechanism holds the ultrasound probe and moves the ultrasound probe while a surface of the ultrasound probe is directed toward a subject. Processing circuitry detects, based on the ultrasound wave, distance information between a body surface and the surface of the ultrasound probe, with respect to a first scan position and a second scan position set along the body surface. The processing circuitry controls ultrasound scans performed in the first scan position and in the second scan position by the ultrasound probe moved by the mechanical mechanism based on the distance information. The processing circuitry controls the mechanical mechanism so as to move the ultrasound probe to the second scan position after the distance information detection and the ultrasound scan in the first scan position are performed.

### CROSS-REFERENCE TO RELATED APPLICATIONS

This application is based upon and claims the benefit of priority from Japanese Patent Application No. 2019-050609, filed on Mar. 19, 2019, the entire contents of which are incorporated herein by reference. The entire contents of the prior Japanese Patent Application No. 2020-048967, filed on Mar. 19, 2020, are also incorporated herein by reference.

### FIELD

Embodiments described herein relate generally to an ultrasound automatic scanning system, an ultrasound diagnosis apparatus, and an ultrasound scanning support apparatus.

### BACKGROUND

Conventionally, ultrasound diagnosing processes are performed by obtaining information about a tissue structure, a blood flow, or the like from the inside a human body, as a result of a medical technologist or a medical doctor operating an ultrasound probe over the body surface of a subject. For example, in accordance with the diagnosed site or diagnosis specifics, the medical technologist or the medical doctor scans the inside of the subject with ultrasound waves, by operating, over the body surface, the ultrasound probe configured to transmit and receive ultrasound waves, to acquire an ultrasound image exhibiting the tissue structure or an ultrasound image exhibiting the information about the blood flow or the like.

For these ultrasound diagnosing processes, performing a scan by using a robot has been proposed in recent years. For example, a technique is known by which position information of the body surface of a subject is obtained from a photo of the subject taken by a camera, so as to generate a scan path indicating a moving trajectory of an ultrasound probe from the obtained position information, so that a robot moves the ultrasound probe along the generated scan path.

### BRIEF DESCRIPTION OF THE DRAWINGS

FIG. 1 is a block diagram illustrating an exemplary configuration of an ultrasound diagnosis apparatus according to a first embodiment;

FIG. 2 is a drawing of an external appearance illustrating an example of a mechanical mechanism according to the first embodiment;

FIG. 3 is a drawing for explaining an example of a table for a scan target site according to the first embodiment;

FIG. 4A is a drawing illustrating an example of an arm rest according to the first embodiment;

FIG. 4B is a drawing illustrating another example of the arm rest according to the first embodiment;

FIG. 5 is a drawing for explaining an example of an automatic scan performed by the ultrasound diagnosis apparatus according to the first embodiment;

FIG. 6 is a flowchart for explaining a procedure in a process performed by the ultrasound diagnosis apparatus according to the first embodiment;

FIG. 7 is a drawing for explaining an example of an automatic scan performed by an ultrasound diagnosis apparatus according to a second embodiment;

FIG. 8 is a drawing for explaining an example of a process performed by a detecting function according to the second embodiment;

FIG. 9 is a flowchart for explaining a procedure in a process performed by the ultrasound diagnosis apparatus according to the second embodiment;

FIG. 10 is a drawing for explaining an example of an ultrasound probe according to a third embodiment; and

FIG. 11 is a drawing for explaining another example of the ultrasound probe according to the third embodiment.

### DETAILED DESCRIPTION

An ultrasound automatic scanning system according to an embodiment includes at least one ultrasound probe, a mechanical mechanism, and processing circuitry. The one or more ultrasound probes are each configured to transmit and receive an ultrasound wave. The mechanical mechanism is configured to hold the ultrasound probe and to move the ultrasound probe while an ultrasound wave transmission-reception surface of the ultrasound probe is directed toward a subject. The processing circuitry is configured to detect, on a basis of the ultrasound wave transmitted and received by the ultrasound probe, distance information between a body surface of the subject and the ultrasound wave transmission-reception surface of the ultrasound probe, with respect to a first scan position and a second scan position set along the body surface of the subject. The processing circuitry is configured to control the mechanical mechanism so as to move the ultrasound probe on a basis of the distance information. The processing circuitry is configured to control ultrasound scans performed in the first scan position and in the second scan position on the subject by the ultrasound probe moved by the mechanical mechanism. The processing circuitry is further configured to control the mechanical mechanism so as to move the ultrasound probe to the second scan position after the distance information detection and the ultrasound scan in the first scan position are performed.

Exemplary embodiments of an ultrasound automatic scanning system, an ultrasound diagnosis apparatus, and an ultrasound scanning support apparatus of the present disclosure will be explained in detail, with reference to the accompanying drawings. Possible embodiments of the ultrasound automatic scanning system, the ultrasound diagnosis apparatus, and the ultrasound scanning support apparatus of the present disclosure are not limited to the embodiments described below. Further, some of the constituent elements in the explanations below that are the same as one another will be referred to by using the same reference characters, and duplicate explanations thereof will be omitted.

### First Embodiment

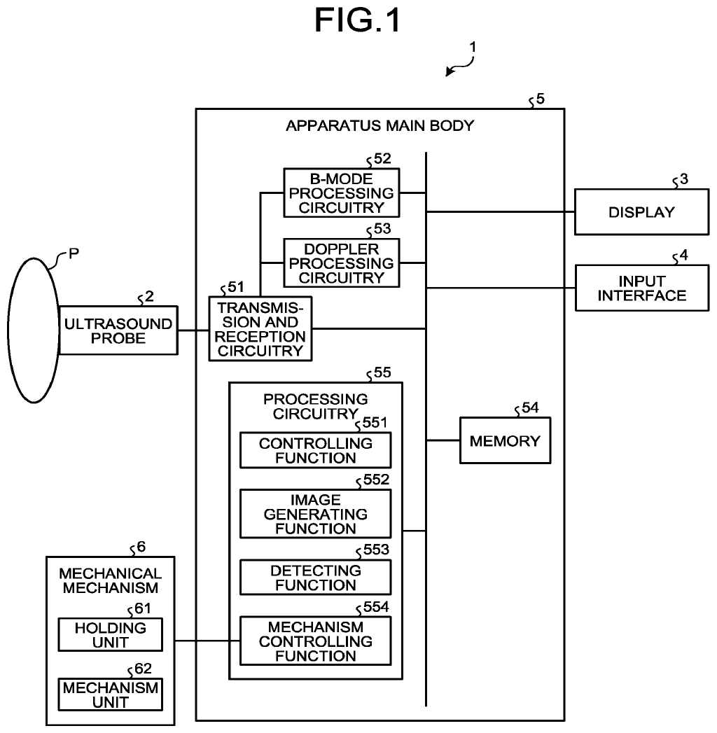

To begin with, an ultrasound diagnosis apparatus according to a first embodiment will be explained. FIG. 1 is a block diagram illustrating an exemplary configuration of an ultrasound diagnosis apparatus 1 according to the first embodiment. As illustrated in FIG. 1, the ultrasound diagnosis apparatus 1 according to the present embodiment includes an ultrasound probe 2, a display 3, an input interface 4, and an apparatus main body 5. The ultrasound probe 2, the display 3, and the input interface 4 are communicably connected to the apparatus main body 5. Further, in the ultrasound diagnosis apparatus 1 according to the present embodiment, a mechanical mechanism 6 is communicably connected to the apparatus main body 5. A structure including the ultrasound diagnosis apparatus 1 and the mechanical mechanism 6 serves as an example of the ultrasound automatic scanning system of the present disclosure.

The ultrasound probe 2 is connected to transmission and reception circuitry 51 included in the apparatus main body 5. For example, the ultrasound probe 2 has a plurality of piezoelectric transducer elements in the probe main body. Each of the plurality of piezoelectric transducer elements is configured to generate an ultrasound wave on the basis of a drive signal supplied thereto from the transmission and reception circuitry 51. Further, the ultrasound probe 2 is configured to receive reflected waves from a subject P and convert the reflected waves into electrical signals. Further, in the probe main body, the ultrasound probe 2 includes a matching layer provided for the piezoelectric transducer elements, as well as a backing member or the like that prevents the ultrasound waves from propagating rearward from the piezoelectric transducer elements. In this situation, the ultrasound probe 2 is detachably connected to the apparatus main body 5. For example, the ultrasound probe 2 is an ultrasound probe of a sector type, a linear type, or a convex type.

When an ultrasound wave is transmitted from the ultrasound probe 2 to the subject P, the transmitted ultrasound wave is repeatedly reflected on a surface of discontinuity of acoustic impedances at a tissue in the body of the subject P and is received as a reflected-wave signal by each of the plurality of piezoelectric transducer elements included in the ultrasound probe 2. The amplitude of the received reflected-wave signal is dependent on the difference between the acoustic impedances on the surface of discontinuity on which the ultrasound wave is reflected. When a transmitted ultrasound pulse is reflected on the surface of a moving blood flow, a cardiac wall, or the like, the reflected-wave signal is, due to the Doppler effect, subject to a frequency shift, depending on a velocity component of the moving members with respect to the ultrasound wave transmission direction.

The present embodiment is applicable to the situation where the subject P is two-dimensionally scanned by using the ultrasound probe 2 represented by a one-dimensional ultrasound probe in which the plurality of piezoelectric transducer elements are arranged in a row and to the situation where the subject P is three-dimensionally scanned by using the ultrasound probe 2 configured to mechanically swing the plurality of piezoelectric transducer elements of a one-dimensional ultrasound probe or the ultrasound probe 2 represented by a two-dimensional ultrasound probe in which the plurality of piezoelectric transducer elements are two-dimensionally arranged in a grid formation.

In the present example, as for the ultrasound probe 2, the probe main body is held by the mechanical mechanism 6, and the ultrasound probe 2 is moved while the ultrasound wave transmission-reception surface thereof is directed toward the subject. Details will be explained later.

The display 3 is configured to display a Graphical User Interface (GUI) used by an operator of the ultrasound diagnosis apparatus 1 for inputting various types of setting requests via the input interface 4 and to display an ultrasound image generated by the apparatus main body 5, and the like. Further, the display 3 is configured to display various types of messages and display information to notify the operator of processing statuses and processing results of the apparatus main body 5. Further, the display 3 has a speaker and is also capable of outputting audio.

The input interface 4 is realized with a trackball, a switch button, a mouse, and a keyboard used for setting a predetermined position (e.g., a region of interest) or the like, a touchpad on which input operations can be performed by touching the operation surface thereof, a touch monitor in which a display screen and a touchpad are integrally formed, a contactless input circuit using an optical sensor, an audio input circuit, and/or the like. The input interface 4 is connected to processing circuitry 55 (explained later) and is configured to convert an input operation received from the operator into an electrical signal and to output the electrical signal to the processing circuitry 55. The input interface 4 of the present disclosure does not necessarily have to include one or more physical operation component parts such as a mouse and a keyboard. For instance, possible examples of the input interface include an electrical signal processing circuit configured to receive an electrical signal corresponding to an input operation from an external input device provided separately from the apparatus and to output the electrical signal to the processing circuitry 55.

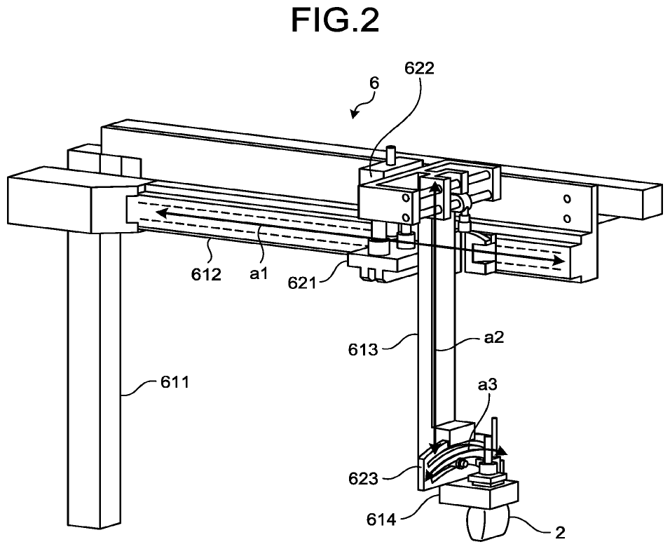

The mechanical mechanism 6 includes: a holding unit 61 configured to hold the probe main body of the ultrasound probe 2; and a mechanism unit 62 configured to move the ultrasound probe 2 to a desired position on the body surface of the subject. In other words, the mechanical mechanism 6 is configured to move the ultrasound probe 2 held by the holding unit 61 to the desired position with movement of the mechanism unit 62. For example, in accordance with control exercised by the apparatus main body 5, the mechanical mechanism 6 moves the ultrasound probe 2. An example of the mechanical mechanism 6 will be explained below, with reference to FIG. 2. The mechanical mechanism 6 illustrated in FIG. 2 is merely an example, and possible embodiments are not limited to this example.

FIG. 2 is a drawing of an external appearance illustrating the example of the mechanical mechanism 6 according to the first embodiment. As illustrated in FIG. 2, the mechanical mechanism 6 includes: the holding unit 61 having a first holding unit 611, a second holding unit 612, a third holding unit 613, and a fourth holding unit 614; and the mechanism unit 62 having a first mechanism unit 621, a second mechanism unit 622, and a third mechanism unit 623. The holding unit 61 is cast by using aluminum or the like and has an adjoining unit configured to adjoin any of the holding units together, an engagement unit configured to be engaged with the mechanism unit 62, a probe holder configured to hold the ultrasound probe 2, and the like. Further, the mechanism unit 62 has a driving unit realized with a motor, an actuator or the like, and an engagement unit configured to be engaged with the holding units or the like.

For example, one end of the first holding unit 611 in terms of the longitudinal direction is adjoined with a basal part (not illustrated) supporting the entirety of the mechanical mechanism 6, whereas the other end is adjoined with the second holding unit 612. As a result, the first holding unit 611 supports all the members that are directly or indirectly held by the second holding unit 612. One end of the second holding unit 612 in terms of the longitudinal direction is adjoined with the first holding unit 611, while the second holding unit 612 is engaged with the first mechanism unit 621 so that the first mechanism unit 621 is slidable along the longitudinal direction. For example, the second holding unit 612 has, along the longitudinal direction, a rail engaged with the first mechanism unit 621, to hold the first mechanism unit 621 on the rail so as to be slidable.

In the present example, as illustrated in FIG. 2, as a result of the second holding unit 612 being adjoined with the first holding unit 611 while the longitudinal direction of the second holding unit 612 corresponds to the horizontal direction, the first mechanism unit 621 moves by sliding in the horizontal direction indicated with the arrow a1. The first mechanism unit 621 is engaged with and held by the second holding unit 612 and moves along the longitudinal direction of the second holding unit 612 due to the drive force of a driving unit such as a motor or an actuator. For example, the first mechanism unit 621 is engaged with the rail of the second holding unit 612 and configured move by sliding on the rail due to the drive force of the driving unit under control of the apparatus main body 5. Further, the first mechanism unit 621 is adjoined with the second mechanism unit 622.

As a result of being adjoined with the first mechanism unit 621, the second mechanism unit 622 is held by the second holding unit 612. Further, the second mechanism unit 622 is engaged with the third holding unit 613 and holds the third holding unit 613, so that the third holding unit 613 is slidable. In other words, the second mechanism unit 622 is configured to move along the longitudinal direction of the second holding unit 612 in conjunction with the sliding movement of the first mechanism unit 621 and is also configured to cause the third holding unit 613 to slide. In this situation, the second mechanism unit 622 causes the third holding unit 613 to slide in a direction orthogonal to the moving direction of the first mechanism unit 621. For example, the second mechanism unit 622 causes the third holding unit 613 to slide in the vertical direction indicated with the arrow a2, by using the drive force of a driving unit based on the control of the apparatus main body 5.

One end of the third holding unit 613 in terms of the longitudinal direction is engaged with the second mechanism unit 622, so that the third holding unit 613 slides on the second mechanism unit 622. For example, the third holding unit 613 has, along the longitudinal direction, a rail engaged with the second mechanism unit 622 and is configured to slide on the second mechanism unit 622 in the vertical direction indicated with the arrow a2. Further, the other end of the third holding unit 613 is engaged with the third mechanism unit 623. In this situation, the third holding unit 613 holds the third mechanism unit 623 so that the third mechanism unit 623 is able to make a rotational movement while using the longitudinal direction of the second holding unit 612 as the axis. For example, the third holding unit 613 holds the third mechanism unit 623 so that the third mechanism unit 623 is able to make the rotational movement in the direction indicated with the arrow a3.

The third mechanism unit 623 is held by the third holding unit 613 as being engaged with the third holding unit 613. Further, the third mechanism unit 623 is adjoined with the fourth holding unit 614. For example, the third mechanism unit 623 makes a rotational movement in the direction indicated with the arrow a3, while holding the fourth holding unit 614 (while the orientation of the fourth holding unit 614 is maintained) with the drive force of a driving unit based on the control of the apparatus main body 5. As a result, the third mechanism unit 623 is able to change the angle of the ultrasound probe 2 held by the fourth holding unit 614.

The fourth holding unit 614 is adjoined with the third mechanism unit 623 and is configured to hold the ultrasound probe 2. For example, as illustrated in FIG. 2, the fourth holding unit 614 holds the ultrasound probe 2, so that the direction of the plane of the ultrasound wave transmission-reception surface of the ultrasound probe 2 is orthogonal to the longitudinal direction of the third holding unit 613.

As explained above, the mechanical mechanism 6 is able to move the ultrasound probe 2 held by the fourth holding unit 614 in the directions indicated with the arrows a1, a2, and a3, with the movements realized by the first mechanism unit 621, the second mechanism unit 622, and the third mechanism unit 623. In other words, the mechanical mechanism 6 is able to move the ultrasound probe 2 in the horizontal directions and the vertical directions and to change the angle of the ultrasound probe 2. Further, the mechanical mechanism 6 illustrated in FIG. 2 is merely an example of mechanical mechanisms, and possible configurations of mechanical mechanisms are not limited to the one illustrated in the drawing. For instance, the mechanical mechanism 6 may include a mechanism unit that moves the ultrasound probe 2 in the direction that is orthogonal to the arrow a1 and is also orthogonal to the arrow a2.

Returning to the description of FIG. 1, the apparatus main body 5 includes the transmission and reception circuitry 51, B-mode processing circuitry 52, Doppler processing circuitry 53, a memory 54, and the processing circuitry 55. In the ultrasound diagnosis apparatus 1 illustrated in FIG. 1, processing functions are stored in the memory 54, in the form of computer-executable programs. The transmission and reception circuitry 51, the B-mode processing circuitry 52, the Doppler processing circuitry 53, and the processing circuitry 55 are processors configured to realize the functions corresponding to the programs, by reading and executing the programs from the memory 54. In other words, the circuits that have read the programs have the functions corresponding to the read programs.

The transmission and reception circuitry 51 includes a pulse generator, a transmission delay circuit, a pulser, and the like and is configured to supply the drive signal to the ultrasound probe 2. The pulse generator is configured to repeatedly generate a rate pulse for forming a transmission ultrasound wave at a predetermined rate frequency. Further, the transmission delay circuit is configured to apply a delay time period that is required to converge the ultrasound waves generated by the ultrasound probe 2 into the form of a beam and to determine transmission directionality and that corresponds to each of the piezoelectric transducer elements, to each of the rate pulses generated by the pulse generator. Further, the pulser is configured to apply the drive signal (a drive pulse) to the ultrasound probe 2 with timing based on the rate pulses. In other words, by varying the delay time periods applied to the rate pulses, the transmission delay circuit is able to arbitrarily adjust the transmission directions of the ultrasound waves transmitted from the surfaces of the piezoelectric transducer elements.

The transmission and reception circuitry 51 has a function that is able to instantly change transmission frequencies, transmission drive voltage, and the like, for the purpose of executing a predetermined scan sequence on the basis of an instruction from the processing circuitry 55 (explained later). In particular, the function to change the transmission drive voltage is realized by using a linear-amplifier-type transmission circuit of which the value can be instantly switched or by using a mechanism configured to electrically switch between a plurality of power source units.

Further, the transmission and reception circuitry 51 includes a pre-amplifier, an Analog-to-Digital (A/D) converter, a reception delay circuit, an adder, and the like and is configured to generate reflected-wave data by performing various types of processes on the reflected-wave signals received by the ultrasound probe 2. The pre-amplifier is configured to amplify the reflected-wave signal for each of the channels. The A/D converter is configured to perform an A/D conversion on the amplified reflected-wave signals. The reception delay circuit is configured to apply a delay time period required to determine reception directionality. The adder is configured to generate the reflected-wave data by performing an adding process on the reflected-wave signals processed by the reception delay circuit. As a result of the adding process performed by the adder, reflected components of the reflected-wave signals that are from the direction corresponding to the reception directionality are emphasized, so that a comprehensive beam used in the ultrasound wave transmission and reception is formed on the basis of the reception directionality and the transmission directionality.

The B-mode processing circuitry 52 is configured to generate data (B-mode data) in which the signal intensity is expressed by a degree of brightness, by receiving the reflected-wave data from the transmission and reception circuitry 51 and performing a logarithmic amplification, an envelope detecting process, and/or the like thereon.

The Doppler processing circuitry 53 is configured to generate data (Doppler data) obtained by extracting moving member information such as velocity, dispersion, power, and the like with respect to multiple points, by performing a frequency analysis to obtain velocity information from the reflected-wave data received from the transmission and reception circuitry 51 and extracting a blood flow, a tissue, and a contrast agent echo component subject to the Doppler effect. For example, the moving members are fluids such as blood flowing in blood vessels, lymph flowing in lymphatic ducts, and the like.

The B-mode processing circuitry 52 and the Doppler processing circuitry 53 are each capable of processing both two-dimensional reflected-wave data and three-dimensional reflected-wave data. In other words, the B-mode processing circuitry 52 is configured to generate two-dimensional B-mode data from two-dimensional reflected-wave data and to generate three-dimensional B-mode data from three-dimensional reflected-wave data. Further, the Doppler processing circuitry 53 is configured to generate two-dimensional Doppler data from two-dimensional reflected-wave data and to generate three-dimensional Doppler data from three-dimensional reflected-wave data. The three-dimensional B-mode data is data in which a brightness value is assigned in correspondence with the intensity of reflection from a reflection source positioned at each of a plurality of points (sampling points) set on scanning lines within a three-dimensional scanned range. Further, the three-dimensional Doppler data is data in which a brightness value is assigned in correspondence with a value of blood flow information (velocity, dispersion, and power) to each of a plurality of points (sampling points) set on the scanning lines within the three-dimensional scanned range.

The memory 54 is configured to store therein display-purpose image data generated by the processing circuitry 55. Further, the memory 54 is also capable of storing therein any of the data generated by the B-mode processing circuitry 52 and the Doppler processing circuitry 53. Further, the memory 54 is also configured to store therein control programs for performing the ultrasound wave transmission and reception, image processing processes, and display processes, as well as various types of data such as diagnosis information (e.g., patients' IDs, medical doctors' observations), diagnosis protocols, various types of body marks, and the like.

The processing circuitry 55 is configured to control the entirety of processes performed by the ultrasound diagnosis apparatus 1. More specifically, the processing circuitry 55 performs various types of processes by reading and executing programs corresponding to a controlling function 551, an image generating function 552, a detecting function 553, and a mechanism controlling function 554 illustrated in FIG. 1, from the memory 54. In this situation, the controlling function 551 is an example of a scan controlling unit. The detecting function 553 is an example of a detecting unit. The mechanism controlling function 554 is an example of a controlling unit.

For example, the processing circuitry 55 is configured to control processes performed by the transmission and reception circuitry 51, the B-mode processing circuitry 52, and the Doppler processing circuitry 53, on the basis of the various types of setting requests input from the operator via the input interface 4, as well as various types of control programs and various types of data read from the memory 54. Further, the processing circuitry 55 is configured to exercise control so that the display 3 displays the display-purpose ultrasound image data (which hereinafter may be referred to as ultrasound images) stored in the memory 54. Further, the processing circuitry 55 is configured to exercise control so that the display 3 displays processing results. For example, the processing circuitry 55 controls the entirety of the apparatus and controls the processes described above, by reading and executing the programs corresponding to the controlling function 551.

The image generating function 552 is configured to generate the ultrasound image data from the data generated by the B-mode processing circuitry 52 and the Doppler processing circuitry 53. In other words, the image generating function 552 is configured to generate B-mode image data in which the intensities of the reflected waves are expressed as brightness levels, from the two-dimensional B-mode data generated by the B-mode processing circuitry 52. The B-mode image data is data rendering the shape of a tissue in the region subject to an ultrasound scan. Further, the image generating function 552 is configured to generate Doppler image data indicating the moving member information, from the two-dimensional Doppler data generated by the Doppler processing circuitry 53. The Doppler image data is velocity image data, dispersion image data, power image data, or image data combining together any of these types of image data. The Doppler image data is data indicating fluid information related to the fluid flowing through the region subject to an ultrasound scan.

In this situation, generally speaking, the image generating function 552 converts (by performing a scan convert process) a scanning line signal sequence from an ultrasound scan into a scanning line signal sequence in a video format used by, for example, television and generates the display-purpose ultrasound image data. More specifically, the image generating function 552 generates the display-purpose ultrasound image data by performing a coordinate transformation process compliant with the ultrasound scanning mode used by the ultrasound probe 2. Further, as various types of image processing processes besides the scan convert process, the image generating function 552 performs, for example, an image processing process (a smoothing process) to re-generate an average brightness value image, an image processing process (an edge enhancement process) that uses a differential filter inside an image, or the like, by using a plurality of image frames resulting from the scan convert process. Also, the image generating function 552 combines text information of various types of parameters, scale graduations, body marks, and the like with the ultrasound image data.

In other words, the B-mode data and the Doppler data are each ultrasound image data before the scan convert process. The data generated by the image generating function 552 is the display-purpose ultrasound image data after the scan convert process. The B-mode data and the Doppler data may be referred to as raw data.

Further, the image generating function 552 is configured to generate three-dimensional B-mode image data by performing a coordinate transformation process on the three-dimensional B-mode data generated by the B-mode processing circuitry 52. Further, the image generating function 552 is configured to generate three-dimensional Doppler image data by performing a coordinate transformation process on the three-dimensional Doppler data generated by the Doppler processing circuitry 53. The three-dimensional B-mode data and the three-dimensional Doppler data are each volume data before the scan convert process. In other words, the image generating function 552 generates “the three-dimensional B-mode image data and the three-dimensional Doppler image data” as “volume data represented by three-dimensional ultrasound image data”.

Further, the image generating function 552 is also capable of performing a rendering process on the volume data, for the purpose of generating various types of two-dimensional image data used for displaying the volume data on the display 3. The detecting function 553 is configured to detect distance information between the ultrasound wave transmission-reception surface of the ultrasound probe 2 and the body surface of the subject. The mechanism controlling function 554 is configured to control the mechanical mechanism 6. Details of the processes performed by the detecting function 553 and the mechanism controlling function 554 will be explained later.

An overall configuration of the ultrasound diagnosis apparatus 1 according to the first embodiment has thus been explained. The ultrasound diagnosis apparatus 1 according to the first embodiment structured as described above makes it possible to improve efficiency of the scans using a robot. More specifically, the ultrasound diagnosis apparatus 1 according to the first embodiment makes it possible to improve efficiency of the scans using a robot without generating a scan path, by performing a probe position determining process and an ultrasound image acquiring process while moving the ultrasound probe 2.

As explained above, for ultrasound diagnosing processes, a technique has been proposed in recent years by which a scan path used by a robot to move an ultrasound probe is generated, so as to perform a scan by moving the ultrasound probe along the generated scan path. According to this technique, however, because the scan path is generated at first, it takes time and there is a certain limitation to the improvement of the efficiency. Further, because it takes time, it is not possible to perform the scan with an optimal scan path when the subject happens to move during the scan, and the quality of the image may be degraded. To cope with these circumstances, the ultrasound diagnosis apparatus 1 according to the first embodiment makes it possible to improve efficiency of the scans using the robot without the need to generate a scan path, by performing the probe position determining process and the ultrasound image acquiring process while moving the ultrasound probe 2.

Next, details of the ultrasound diagnosis apparatus 1 according to the first embodiment will be explained. The detecting function 553 is configured to detect distance information between the body surface of the subject and the ultrasound wave transmission-reception surface of the ultrasound probe, on the basis of an ultrasound wave transmitted and received by the ultrasound probe 2. More specifically, the detecting function 553 is configured to detect the distance information between the body surface of the subject and the ultrasound wave transmission-reception of the ultrasound probe 2, on the basis of reflected-wave data acquired by moving the ultrasound probe 2 kept out of contact with the subject. In other words, the detecting function 553 is configured to detect the distance information between the body surface of the subject and the ultrasound wave transmission-reception surface, on the basis of the reflected-wave data obtained as a result of the ultrasound wave transmitted from the ultrasound probe 2 kept out of contact with the subject being reflected on the body surface of the subject.

The mechanism controlling function 554 is configured to control movements of the mechanism unit 62, by transmitting control signals to driving units included in the mechanism unit 62 of the mechanical mechanism 6. For example, by transmitting a control signal to the first mechanism unit 621, the mechanism controlling function 554 controls the driving unit of the first mechanism unit 621 to control the sliding movement of the first mechanism unit 621 in the direction indicated with the arrow a1. Further, for example, by transmitting a control signal to the second mechanism unit 622, the mechanism controlling function 554 controls the driving unit of the second mechanism unit 622 to control the sliding movement of the third holding unit 613 in the direction indicated with the arrow a2. Also, for example, by transmitting a control signal to the third mechanism unit 623, the mechanism controlling function 554 controls the driving unit of the third mechanism unit 623 to control the rotational movement of the third mechanism unit 623 in the direction indicated with the arrow a3. Further, the mechanism controlling function 554 transmits control information to the detecting function 553.

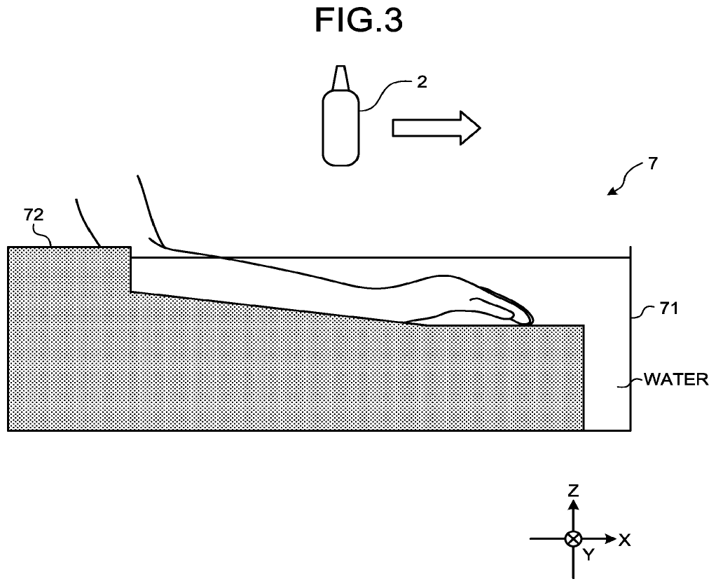

In this situation, in the ultrasound diagnosis apparatus 1 according to the first embodiment, as described above, while the mechanical mechanism 6 is moving the ultrasound probe 2 kept out of contact with the subject, the reflected-wave data of the site subject to the scan (hereinafter, “scan target site”) is acquired. Accordingly, in the first embodiment, water is used as an acoustic medium so that it is possible to transmit and receive ultrasound waves even while the ultrasound probe 2 is kept out of contact with the body surface of the subject. FIG. 3 is a drawing for explaining an example of a table 7 for a scan target site according to the first embodiment. FIG. 3 illustrates the example of the table used when the scan target site is a section from the antebrachial region to the hand.

For example, as illustrated in FIG. 3, the table 7 has a water tank 71 and an arm rest 72. The water tank 71 contains water. The arm rest 72 has an elbow rest part and a slope part that gradually slopes downward from the elbow rest part. In this situation, as illustrated in FIG. 3, the table 7 is configured with the water tank 71 containing water so that when the arm is placed on the arm rest 72, the section from the antebrachial region to the hand is submerged in the water. Further, the mechanical mechanism 6 is arranged so that the ultrasound probe 2 is movable in the direction indicated with the arrow in FIG. 3 with respect to the arm placed on the table 7. For example, the mechanical mechanism 6 is arranged so that the longitudinal direction of the second holding unit 612 illustrated in FIG. 2 is parallel to the longitudinal direction of the antebrachial region placed on the table 7 and so that the scan target site is scanned by ultrasound waves transmitted from the ultrasound probe 2 held by the fourth holding unit 614.

Further, to scan the scan target site, the ultrasound probe 2 is put into the water in the water tank 71 and the ultrasound wave is transmitted and received while the ultrasound probe 2 is kept out of contact with the body surface of the scan target site. For example, the third holding unit 613 is caused to slide in the vertical direction by the driving of the second mechanism unit 622, so that the ultrasound probe 2 is in the water of the water tank 71 while the ultrasound probe 2 is kept out of contact with the body surface. In the following sections, as illustrated in FIG. 3, the moving direction of the ultrasound probe 2 may be referred to as an X-direction, while the direction orthogonal and parallel to the X-direction may be referred to as a Y-direction, and the direction orthogonal to the X-direction and to the Y-direction may be referred to as a Z-direction.

In this situation, to stabilize the placement of the scan target site, the table 7 may be provided with a recess or a projection in the arm rest 72. In other words, the arm rest 72 may be provided with the recess or the projection for fixing the scan target site so that, when subjects place the scan target sites on the table 7, the scan target sites are always arranged in substantially the same position with respect to the table 7.

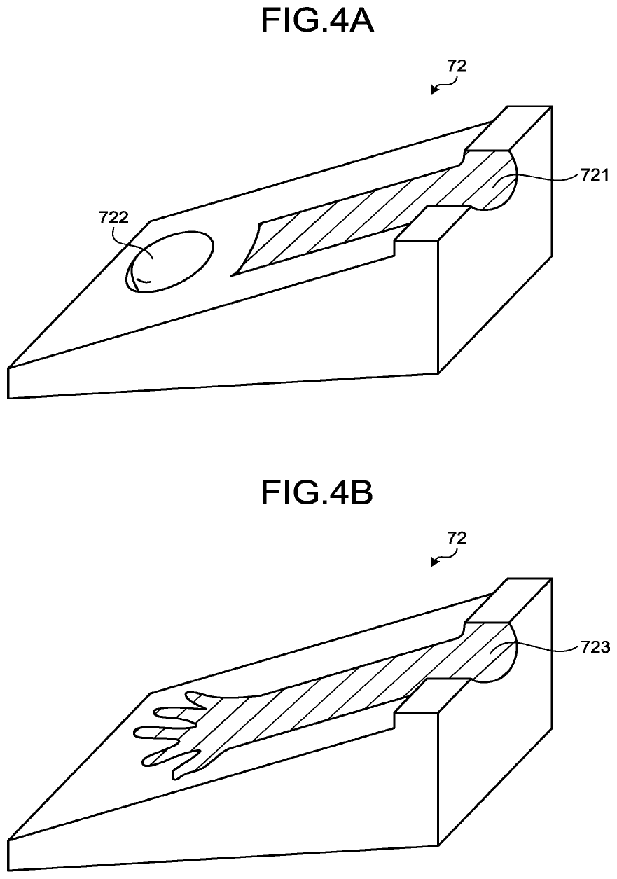

FIGS. 4A and 4B are drawings illustrating examples of the arm rest 72 according to the first embodiment. For example, as illustrated in FIG. 4A, the arm rest 72 has: a recess part 721 which extends from an elbow rest part to the slope part and on which the section of the arm from the elbow to the antebrachial region is placed; and a projection part 722 that supports the hand. By using the arm rest 72 structured in this manner, for example, subjects are always able to place their arms substantially in the same position, by placing the section of the arm from the elbow to the antebrachial region in the recess part 721 and lightly gripping the projection part 722.

In another example, as illustrated in FIG. 4B, the arm rest 72 may have a recess part 723 which extends from the elbow rest part to the slope part and on which the section of the arm from the elbow to the hand is placed. In this situation, the recess part 723 is recessed in the shape of the human hand so that, for example, subjects are always able to place their arms substantially in the same position by placing the section of their arms from the elbow to the hand in the recess part 723.

In the first embodiment, while having the arm placed on the table 7 structured as described above, the mechanical mechanism 6 performs the scan in a contactless manner. More specifically, the detecting function 553 is configured to detect the distance information between the body surface of the subject and the ultrasound wave transmission-reception surface of the ultrasound probe 2, with respect to at least one scan position set along the direction in which the ultrasound probe 2 is moved by the mechanical mechanism 6. Every time distance information is detected by the detecting function 553, the mechanism controlling function 554 is configured to control the mechanical mechanism 6 so as to move the ultrasound probe 2 to a probe position corresponding to at least one scan position derived on the basis of the detected distance information. Further, the controlling function 551 is configured to exercise control so that an ultrasound image is acquired in the probe position.

For example, in the first embodiment, the subject at first places the scan target site on the table. In this situation, when the scan target site is an arm, the subject places the arm on the table 7 described above. Further, the mechanism controlling function 554 controls the mechanism unit 62 of the mechanical mechanism 6, so as to move the ultrasound probe 2 to an initial position. In this situation, for example, the initial position may be determined by a user such as the operator or may be determined in advance for each table.

For example, when the initial position is determined by the user, the operator has the ultrasound probe 2 moved to the initial position, by causing the mechanism controlling function 554 to control movements of the ultrasound probe 2 via the input interface 4. In another example, when the initial position is determined in advance for each table, the mechanism controlling function 554 identifies the initial position of the currently-used table on the basis of information indicating a correspondence relationship between tables and initial positions and further controls the mechanism unit 62 of the mechanical mechanism 6 so as to move the ultrasound probe 2 to the identified initial position. In this situation, the information indicating the correspondence relationship between tables and initial positions has, for example, the initial positions set in correspondence with the tables while taking into consideration the sizes and the shapes of the tables, and the information is stored in the memory 54 in advance.

In this situation, the initial position is such a position where the ultrasound probe 2 is in the water of the water tank 71, while the ultrasound probe 2 is kept out of contact with the body surface. For example, the initial position is such a position where the ultrasound probe 2 is in the water on the elbow rest side of the arm rest 72, while the ultrasound probe 2 is kept out of contact with the body surface.

Further, when the ultrasound probe 2 has been arranged in the initial position, the controlling function 551, the detecting function 553, and the mechanism controlling function 554 move the position of the ultrasound probe 2 while measuring the distance between the body surface of the subject and the ultrasound wave transmission-reception surface of the ultrasound probe 2 and exercise control so that ultrasound images are acquired in scan positions set along the moving direction.

More specifically, on the basis of the ultrasound wave transmitted and received by the ultrasound probe 2, the detecting function 553 is configured to detect the distance information between the body surface of the subject and the ultrasound wave transmission-reception surface of the ultrasound probe, with respect to a first scan position and a second scan position set along the body surface of the subject. In other words, on the basis of an ultrasound wave transmitted and received after the ultrasound probe 2 is moved by the mechanism controlling function 554 to a scan position set along the moving direction, the detecting function 553 detects, with respect to the scan position, the distance between the body surface of the subject and the ultrasound wave transmission-reception surface of the ultrasound probe 2. Further, every time the distance is detected by the detecting function 553, the mechanism controlling function 554 moves the ultrasound probe 2 to a probe position (the position of the ultrasound probe 2 with respect to the body surface of the subject) based on the distance information. In the first scan position and the second scan position, the controlling function 551 controls the ultrasound scans performed on the subject by the ultrasound probe moved to the probe position by the mechanical mechanism 6. After the ultrasound image is acquired in the probe position, the mechanism controlling function 554 moves the ultrasound probe 2 to another scan position set along the moving direction. In other words, after the distance information detection and the ultrasound scan are performed in the first scan position, the mechanism controlling function 554 further controls the mechanical mechanism 6 so as to move the ultrasound probe 2 to the second scan position. In this situation, the probe position derived on the basis of the distance information is, for example, a position where the focal point of the ultrasound wave falls within a region of interest. Further, for example, the region of interest is set in advance on the basis of the distance from the body surface or the like.

In other words, in the ultrasound diagnosis apparatus 1 according to the first embodiment, it is possible to cause the robot to perform the scan in the optimal probe position without generating a scan path, by repeatedly performing the following processes: moving the ultrasound probe 2 to a scan position set along the moving direction of the ultrasound probe 2; measuring the distance between the ultrasound wave transmission-reception surface of the ultrasound probe 2 and the body surface; moving the ultrasound probe 2 to the probe position derived on the basis of the result of measuring the distance; and acquiring an ultrasound image in the probe position.

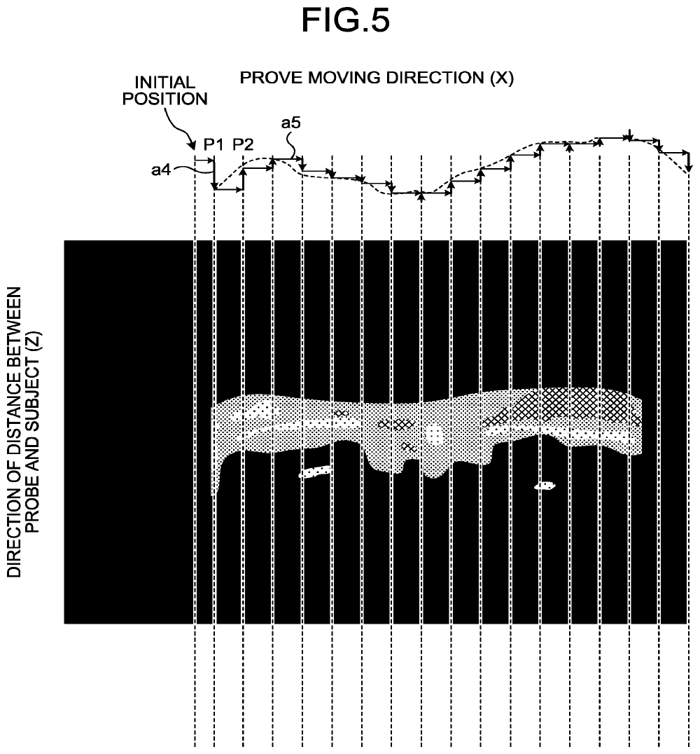

FIG. 5 is a drawing for explaining an example of the automatic scan performed by the ultrasound diagnosis apparatus 1 according to the first embodiment. In FIG. 5, the horizontal direction expresses the moving direction of the ultrasound probe 2 (the X-direction), whereas the vertical direction expresses the direction of the distance between the ultrasound probe 2 and the subject (the Z-direction). Further, for the sake of convenience in the explanation, FIG. 5 illustrates cross-sectional ultrasound images of the scan target site (the arm) along the moving direction of the ultrasound probe 2; however, in actuality, the ultrasound images of the scan target site have not yet been acquired.

For example, after the ultrasound probe 2 is moved to the initial position indicated in FIG. 5, the mechanism controlling function 554 controls the mechanical mechanism 6 so as to move the ultrasound probe 2 to a scan position P1 (the first scan position). In one example, by exercising control so that only the driving unit of the first mechanism unit 621 is driven, the mechanism controlling function 554 controls the ultrasound probe 2 to slide in the horizontal direction, without changing the position of the ultrasound probe 2 in the vertical direction. In other words, the mechanism controlling function 554 causes the ultrasound probe 2 to slide along the direction from the antebrachial region to the hand.

In this situation, the scan position is determined on the basis of a scan interval set in advance. In other words, for the scan performed by the robot, the distance from the initial position to the scan position P1 is determined on the basis of the scan interval along the moving direction of the ultrasound probe 2 set in advance. The mechanism controlling function 554 drives the driving unit of the first mechanism unit 621, so that the ultrasound probe 2 moves in the X-direction by the determined distance. In this situation, when spatial resolutions are taken into consideration, it is desirable to arrange the scan interval (the interval distances between the plurality of scan positions set along the body surface of the subject) to be 2 mm or shorter.

When the scan position P1 is reached, the controlling function 551 controls the transmission and reception circuitry 51 so that an ultrasound wave is transmitted from the ultrasound probe 2, and a reflected wave is received. Further, on the basis of the reflected-wave data generated by the transmission and reception circuitry 51, the image generating function 552 generates ultrasound image data. Further, the image generating function 552 transmits the generated ultrasound image data to the detecting function 553.

By using the ultrasound image data received from the image generating function 552, the detecting function 553 calculates the distance between the body surface of the scan target site and the ultrasound wave transmission-reception surface of the ultrasound probe 2. For example, the detecting function 553 detects the body surface from the ultrasound image data taken in the scan position P1 and further calculates the distance from the detected body surface to the ultrasound wave transmission-reception surface. In this situation, the process of detecting the body surface from the ultrasound image data is performed by using an arbitrary existing method. Further, the example was explained above in which the body surface is detected from the ultrasound image data so that the distance is calculated from the detected body surface to the ultrasound wave transmission-reception surface; however, possible embodiments are not limited to this example. For instance, it is also acceptable to calculate a distance, on the basis of the time between the transmission of the ultrasound wave and the reception of the reflected wave, together with the speed of the ultrasound wave in the water.

As explained above, when the distance between the ultrasound probe 2 and the body surface has been detected, the mechanism controlling function 554 controls the mechanical mechanism 6, so as to move the ultrasound probe 2 to a probe position derived on the basis of the detected distance information. In other words, the mechanism controlling function 554 moves the ultrasound probe 2 so that the distance between the body surface of the subject and the ultrasound wave transmission-reception surface with respect to the scan position is equal to the distance based on the distance information. In this situation, the probe position derived on the basis of the distance information is set, as explained above, as a position where the focal point of the ultrasound wave falls within the region of interest. In one example, when a region with a depth of 1 cm from the body surface is set as the region of interest, the probe position is determined to be a position where the focal point of the ultrasound wave falls within the region with the depth of 1 cm from the body surface.

In other words, on the basis of the focal point distance of the ultrasound probe 2 and the distance information detected by the detecting function 553, the mechanism controlling function 554 identifies the position of the focal point at the current point in time in the Z-direction. Further, the mechanism controlling function 554 calculates the moving distance of the ultrasound probe 2 in the Z-direction that allows the identified focal point position to fall within the region of interest and further controls the mechanical mechanism 6 so as to move the ultrasound probe 2 by the calculated moving distance.

For example, on the basis of the focal point distance of the ultrasound probe 2 and the distance information corresponding to the scan position P1 indicated in FIG. 5, the mechanism controlling function 554 calculates the moving distance indicated with the arrow a4 and further controls the mechanical mechanism 6 so as to move the ultrasound probe 2 by the moving distance indicated with the arrow a4. In one example, by exercising control so that only the driving unit of the second mechanism unit 622 is driven, the mechanism controlling function 554 moves the ultrasound probe 2 to the probe position, by causing the ultrasound probe 2 to slide in the Z-direction without changing the position of the ultrasound probe 2 in the X-direction.

When the ultrasound probe 2 has been moved to the probe position, the controlling function 551 controls the transmission and reception circuitry 51 so that an ultrasound wave is transmitted from the ultrasound probe 2, and a reflected wave is received. Further, on the basis of the reflected-wave data generated by the transmission and reception circuitry 51, the image generating function 552 generates ultrasound image data. The image generating function 552 stores the generated ultrasound image data into the memory 54. In this situation, as position information of the ultrasound image data, the image generating function 552 is also capable of storing a driving amount of the mechanical mechanism 6 (e.g., a driving amount for the first mechanism unit 621) so as to be kept in correspondence.

When the controlling function 551 has acquired the ultrasound image data in the scan position P1, the mechanism controlling function 554 controls the mechanical mechanism 6 so as to move the ultrasound probe 2 to a scan position P2 (the second scan position). In this situation, the distance to the scan position P2 is determined on the basis of the scan interval, as explained above.

Further, similarly to the process performed with respect to the scan position P1, the controlling function 551 acquires reflected-wave data, and the detecting function 553 detects distance information with respect to the scan position P2. Further, the mechanism controlling function 554 calculates a moving distance indicated with the arrow a5 and moves the ultrasound probe 2 to the probe position, so that the controlling function 551 acquires ultrasound image data in the scan position P2.

By using the ultrasound diagnosis apparatus 1 according to the first embodiment, it is possible, as explained above, to perform the automatic scan in the optimal scan position (e.g., the position where the focal point of the ultrasound wave falls within the region of interest) without generating a scan path, by successively performing the processes of: detecting the distance information between the body surface of the subject and the ultrasound wave transmission-reception surface of the ultrasound probe 2; moving the ultrasound probe 2 to the probe position; and acquiring the ultrasound image data.

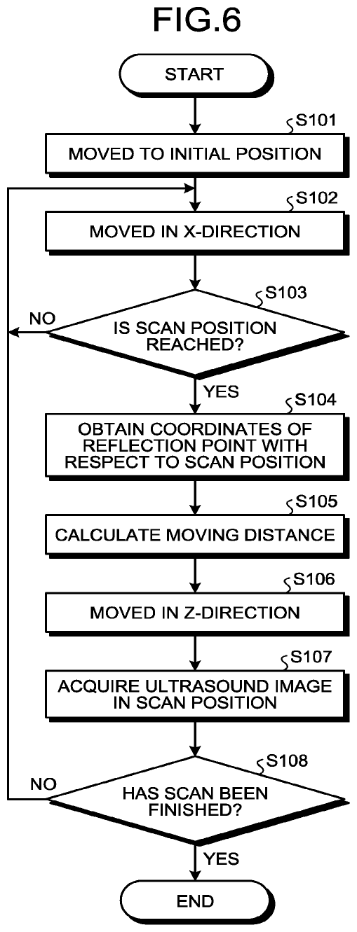

Next, a process performed by the ultrasound diagnosis apparatus 1 according to the first embodiment will be explained, with reference to FIG. 6. FIG. 6 is a flowchart for explaining a procedure in the process performed by the ultrasound diagnosis apparatus 1 according to the first embodiment. In the present example, steps S101 through S103, S105, and S106 in FIG. 6 are steps executed as a result of the processing circuitry 55 reading the program corresponding to the mechanism controlling function 554 from the memory 54. Step S104 is a step executed as a result of the processing circuitry 55 reading the programs corresponding to the controlling function 551 and the detecting function 553 from the memory 54. Steps S107 and S108 are steps executed as a result of the processing circuitry 55 reading the program corresponding to the controlling function 551 from the memory 54.

In the ultrasound diagnosis apparatus 1 according to the first embodiment, the processing circuitry 55 moves the ultrasound probe 2 to the initial position (step S101). Further, the processing circuitry 55 moves the ultrasound probe 2 in the X-direction while keeping the ultrasound probe 2 out of contact with the subject (step S102) and judges whether or not the ultrasound probe 2 has reached a scan position (step S103).

When the ultrasound probe 2 has reached a scan position (step S103: Yes), the processing circuitry 55 obtains the coordinates of the ultrasound wave reflection point with respect to the scan position (step S104). In other words, the processing circuitry 55 detects distance information between the ultrasound probe 2 and the body surface of the subject, with respect to the scan position. In this situation, until the scan position is reached (step S103: No), the processing circuitry 55 moves the ultrasound probe 2 in the X-direction.

Subsequently, the processing circuitry 55 calculates the moving distance in the Z-direction with respect to the scan position (step S105) and moves the ultrasound probe 2 in the Z-direction (step S106) to arrange the ultrasound probe 2 in a probe position. After that, the processing circuitry 55 acquires an ultrasound image in the scan position (step S107) and judges whether or not the scan has been finished (step S108).

When the scan has not been finished, the processing circuitry 55 returns to step S102 and continues the process. On the contrary, when the scan has been finished, the process ends. The judgment as to whether or not the scan has been finished, for example, may be made on the basis of an operation performed by the user via the input interface 4 or may be made on the basis of distance information detected by the detecting function 553. When the judgment is made on the basis of the distance information, for example, the processing circuitry 55 may determine that the position of the ultrasound probe 2 is outside of the scan target site when the difference between the distance information detected with respect to the post-move scan position and the distance information detected with respect to the immediately-preceding scan position becomes equal to or larger than a threshold value and may thus determine that the scan has been finished.

As explained above, according to the first embodiment, the ultrasound probe 2 is configured to transmit and receive the ultrasound wave. The mechanical mechanism 6 is configured to hold the ultrasound probe 2 and to move the ultrasound probe 2 while the ultrasound wave transmission-reception surface of the ultrasound probe 2 is directed toward the subject. On the basis of the ultrasound wave transmitted and received by the ultrasound probe 2, the detecting function 553 is configured to detect the distance information between the body surface of the subject and the ultrasound wave transmission-reception surface of the ultrasound probe 2. The mechanism controlling function 554 is configured to control the mechanical mechanism 6 so as to move the ultrasound probe 2 to the probe position based on the distance information. The process performed by the detecting function 553 to detect the distance information and the process performed by the mechanism controlling function 554 to control the mechanical mechanism 6 are successively performed. Consequently, the ultrasound diagnosis apparatus 1 according to the first embodiment is able to perform the automatic scan without generating a scan path and makes it possible to improve efficiency of the scan using the robot by shortening the time period required by the scan.

In other words, on the basis of the ultrasound wave transmitted and received by the ultrasound probe 2, the detecting function 553 is configured to detect the distance information between the body surface of the subject and the ultrasound wave transmission-reception surface of the ultrasound probe 2, with respect to the first scan position and the second scan position set along the body surface of the subject. The mechanism controlling function 554 is configured to control the mechanical mechanism 6 so as to move the ultrasound probe 2 on the basis of the distance information. In the first scan position and the second scan position, the controlling function 551 is configured to control the ultrasound scans performed on the subject by the ultrasound probe 2 moved by the mechanical mechanism 6. After the distance information detection and the ultrasound scan are performed in the first scan position, the mechanism controlling function 554 is configured to further control the mechanical mechanism 6 so as to move the ultrasound probe 2 to the second scan position. In this manner, by sequentially performing the distance measuring process and the ultrasound scan for each of the scan positions, the ultrasound diagnosis apparatus 1 according to the first embodiment is able to perform the automatic scan adaptively, even when the position of the subject changes due to body movements, for example.

Further, according to the first embodiment, the detecting function 553 is configured to detect the distance information between the body surface of the subject and the ultrasound wave transmission-reception surface of the ultrasound probe 2, with respect to at least one scan position set along the moving direction of the ultrasound probe 2 by the mechanical mechanism 6. Every time distance information is detected by the detecting function 553, the mechanism controlling function 554 is configured to control the mechanical mechanism 6 so as to move the ultrasound probe 2 to the probe position corresponding to at least one scan position derived on the basis of the detected distance information. Consequently, the ultrasound diagnosis apparatus 1 according to the first embodiment makes it possible to perform the automatic scan without generating a scan path.

Further, according to the first embodiment, on the basis of the ultrasound wave transmitted and received after the ultrasound probe 2 is moved by the mechanism controlling function 554 to the scan position set along the moving direction, the detecting function 553 is configured to detect, with respect to the scan position, the distance information between the body surface of the subject and the ultrasound wave transmission-reception surface of the ultrasound probe 2. Every time distance information is detected by the detecting function 553, the mechanism controlling function 554 moves the ultrasound probe 2 to a probe position based on the distance information and, after an ultrasound image is acquired in the probe position, moves the ultrasound probe 2 to a scan position set along the moving direction. In other words, the mechanism controlling function 554 is configured to move the ultrasound probe 2 so that the distance between the body surface of the subject and the ultrasound wave transmission-reception surface with respect to the scan position is equal to the distance based on the distance information. Further, after the ultrasound scan is performed by the controlling function 551, the mechanism controlling function 554 moves the ultrasound probe 2 to the next scan position set along the body surface of the subject. Consequently, the ultrasound diagnosis apparatus 1 according to the first embodiment makes it possible to successively perform the following processes: detecting the distance information between the body surface of the subject and the ultrasound wave transmission-reception surface of the ultrasound probe 2; moving the ultrasound probe 2 to a probe position; and acquiring ultrasound image data.

Further, according to the first embodiment, the probe position is derived from the distance information so as to be a position where the focal point of the ultrasound wave falls within the region of interest. Consequently, the ultrasound diagnosis apparatus 1 according to the first embodiment makes it possible to perform the automatic scan in the optimal position.

Further, according to the first embodiment, the controlling function 551 is configured to exercise control so that the ultrasound image is acquired in the probe position. Consequently, the ultrasound diagnosis apparatus 1 according to the first embodiment makes it possible to acquire the ultrasound image having excellent image quality in the region of interest.

Also, according to the first embodiment, the interval distances between the plurality of scan positions set along the body surface of the subject are each equal to or shorter than 2 mm. Consequently, the ultrasound diagnosis apparatus 1 according to the first embodiment makes it possible to acquire ultrasound images having high spatial resolutions.

### Second Embodiment

In the first embodiment, the example was explained in which the distance information is detected for each of the scan positions. In a second embodiment, an example will be explained in which pieces of distance information are simultaneously detected with respect to a plurality of scan positions. Compared to the first embodiment, the ultrasound diagnosis apparatus 1 according to the second embodiment is different in the processes performed by the controlling function 551, the detecting function 553, and the mechanism controlling function 554. In the following sections, the second embodiment will be explained while a focus is placed on the differences.

When transmitting and receiving the ultrasound wave to detect the distance information, the controlling function 551 according to the second embodiment is configured to transmit and receive the ultrasound wave while varying the direction thereof. More specifically, the controlling function 551 is configured to scan the body surface in positions from the first scan position to the third scan position, by performing a deflection scan in which the ultrasound wave transmission and reception direction is varied.

Further, the detecting function 553 according to the second embodiment is configured to detect the distance information between the body surface of the subject and the ultrasound wave transmission-reception surface of the ultrasound probe 2, with respect to each of a plurality of scan positions set along the moving direction of the ultrasound probe 2. More specifically, in the first scan position, the detecting function 553 detects the distance information between the body surface of the subject and the ultrasound wave transmission-reception surface of the ultrasound probe 2 with respect to the third scan position set along the body surface of the subject. For example, on the basis of an ultrasound wave obtained by performing a deflection scan, the detecting function 553 detects the distance information between the body surface of the subject and the ultrasound wave transmission-reception surface of the ultrasound probe with respect to the third scan position.

Further, every time distance information is detected by the detecting function 553, the mechanism controlling function 554 sequentially moves the ultrasound probe 2 to a probe position corresponding to a scan position based on the distance information with respect to the scan position. More specifically, after the ultrasound scan in the first scan position is performed, the mechanism controlling function 554 moves the ultrasound probe 2 to the third scan position and also moves the ultrasound probe 2 so that the distance between the body surface of the subject and the ultrasound wave transmission-reception surface with respect to the third scan position is equal to a distance based on the distance information corresponding to the third scan position.

Further, the controlling function 551 according to the second embodiment is configured to exercise control so that an ultrasound scan is performed in each of the scan positions for which the distance information was detected. For example, the controlling function 551 controls an ultrasound scan performed in the third scan position.

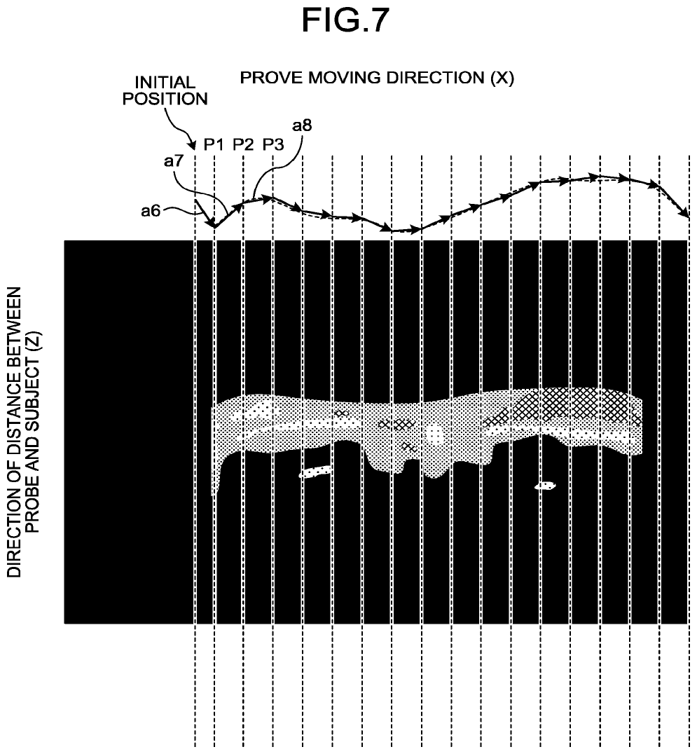

FIG. 7 is a drawing for explaining an example of an automatic scan performed by the ultrasound diagnosis apparatus 1 according to the second embodiment. In FIG. 7, the horizontal direction expresses the moving direction of the ultrasound probe 2 (the X-direction), whereas the vertical direction expresses the direction of the distance between the ultrasound probe 2 and the subject (the Z-direction). Further, for the sake of convenience in the explanation, FIG. 7 illustrates cross-sectional ultrasound images of the scan target site (the arm) along the moving direction of the ultrasound probe 2; however, in actuality, the ultrasound images of the scan target site have not yet been acquired.

According to the second embodiment, for example, after the ultrasound probe 2 is moved to the initial position indicated in FIG. 7, the controlling function 551 controls the transmission and reception circuitry 51 so that an ultrasound wave is transmitted from the ultrasound probe 2, and a reflected wave is received. In this situation, in the second embodiment, by performing a deflection scan in which the ultrasound wave transmission and reception direction is varied, the controlling function 551 scans the body surface of the subject corresponding to a position that is ahead, in terms of the moving direction, of the current position of the ultrasound probe 2.

For example, while the ultrasound probe 2 is arranged in the initial position indicated in FIG. 7, the controlling function 551 transmits and receives an ultrasound wave to and from the body surface corresponding to (that corresponds to the positions of) the scan positions P1 to P3 (the third scan position). On the basis of the reflected-wave data acquired by the controlling function 551, the detecting function 553 is configured to detect the distance between the ultrasound probe 2 and the body surface corresponding to each of the scan positions and further calculates, on the basis of the detected distances, the distance from the ultrasound probe 2 to the body surface corresponding to the time when the ultrasound probe 2 is arranged in each of the scan positions.

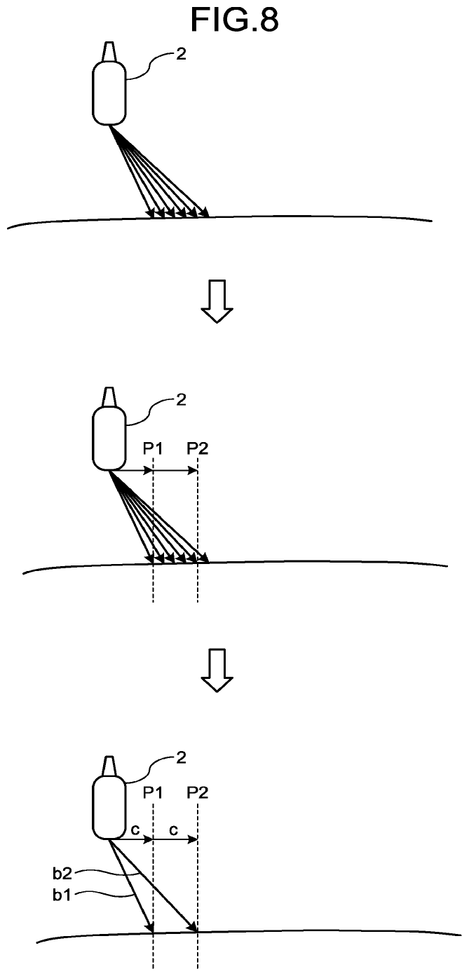

FIG. 8 is a drawing for explaining an example of a process performed by the detecting function 553 according to the second embodiment. FIG. 8 illustrates only the distance calculating processes with respect to the scan position P1 and the scan position P2. For example, as illustrated in the top section of FIG. 8, when the controlling function 551 transmits an ultrasound wave from the ultrasound probe 2 toward the subject while varying the ultrasound wave transmission and reception direction, the detecting function 553 calculates the distance to the body surface of the subject at each of the angles, on the basis of the reflected-wave data from the angle. In other words, the detecting function 553 is configured to detect the position of the body surface of the subject at each of the angles.

Further, for example, as illustrated in the middle section of FIG. 8, the detecting function 553 detects the body surface corresponding to the scan position P1 and to the scan position P2 and further extracts the angle of the reflected-wave data reflected by the detected body surface. After that, by using the distance information corresponding to the extracted angles and the distance in the X-direction from the current position of the ultrasound probe 2 to each of the scan positions, the detecting function 553 calculates the distance between the ultrasound wave transmission-reception surface of the ultrasound probe 2 and the body surface corresponding to the time when the ultrasound probe 2 is arranged in each of the scan positions.

For example, as illustrated in the bottom section of FIG. 8, by using the distance indicated by the arrow b1 and a distance c, the detecting function 553 calculates the distance between the ultrasound wave transmission-reception surface of the ultrasound probe 2 and the body surface corresponding to the time when the ultrasound probe 2 is arranged in the scan position P1. In one example, on the basis of the Pythagorean theorem, the detecting function 553 calculates the distance between the ultrasound wave transmission-reception surface of the ultrasound probe 2 and the body surface corresponding to the time when the ultrasound probe 2 is arranged in the scan position P1. Similarly, by using the distance indicated by the arrow b2 and a distance 2c, the detecting function 553 calculates the distance between the ultrasound wave transmission-reception surface of the ultrasound probe 2 and the body surface corresponding to the time when the ultrasound probe 2 is arranged in the scan position P2.

In this manner, the detecting function 553 simultaneously detects the pieces of distance information with respect to the plurality of scan positions, on the basis of the reflected-wave data acquired through the deflection scan using the ultrasound wave and being performed by the controlling function 551. For example, when the detecting function 553 has detected the distance information corresponding to the scan positions P1 to P3 in FIG. 7, the mechanism controlling function 554 derives the probe position corresponding to each of the scan positions P1 to P3 and further controls the mechanical mechanism 6 so as to move the ultrasound probe 2 to the probe positions.

For example, by driving the driving unit of the first mechanism unit 621 and the driving unit of the second mechanism unit 622, the mechanism controlling function 554 moves the ultrasound probe 2 as indicated by the arrow a6 in FIG. 7. When the ultrasound probe 2 has reached the scan position P1, the controlling function 551 controls the transmission and reception circuitry 51 so that an ultrasound wave is transmitted from the ultrasound probe 2, and a reflected wave is received. After that, on the basis of the reflected-wave data generated by the transmission and reception circuitry 51, the image generating function 552 generates ultrasound image data. The image generating function 552 stores the generated ultrasound image data into the memory 54. In this situation, as position information of the ultrasound image data, the image generating function 552 is also capable of storing a driving amount of the mechanical mechanism 6 (e.g., the driving amount for the first mechanism unit 621) so as to be kept in correspondence.

When the ultrasound image acquisition in the scan position P1 has been finished, the mechanism controlling function 554 moves the ultrasound probe 2 to a probe position corresponding to the scan position P2, as indicated by the arrow a7 in FIG. 7, by driving the driving unit of the first mechanism unit 621 and the driving unit of the second mechanism unit 622. After that, when the ultrasound image acquisition in the scan position P2 has been finished, the mechanism controlling function 554 moves the ultrasound probe 2 to a probe position corresponding to the scan position P3 as indicated by the arrow a8 in FIG. 7, by driving the driving unit of the first mechanism unit 621 and the driving unit of the second mechanism unit 622.

When the ultrasound probe 2 has been moved to the scan position for which the probe position has been derived, the controlling function 551 calculates distance information with respect to the plurality of scan positions, by performing a deflection scan again. In this manner, the ultrasound diagnosis apparatus 1 according to the second embodiment is configured to simultaneously detect the pieces of distance information with respect to the plurality of scan positions and to successively perform the ultrasound image acquisitions in the scan positions.

In the embodiment above, the example was explained in which the controlling function 551 electronically varies the ultrasound wave transmission and reception direction; however, possible embodiments are not limited to this example. For instance, the ultrasound wave transmission and reception direction may be varied by the mechanical mechanism 6 gripping the ultrasound probe 2. In that situation, the mechanical mechanism 6 includes a mechanism unit configured to cause the ultrasound probe 2 to make a rotational movement along the moving direction of the ultrasound probe 2. Further, by driving a driving unit of the mechanism unit, the mechanism controlling function 554 causes the ultrasound probe 2 to make the rotational movement along the moving direction. The controlling function 551 is configured to transmit and receive an ultrasound wave during the rotational movement. The detecting function 553 is configured to detect the positions of the body surface corresponding to the scan positions, on the basis of information about driving amounts of the driving unit (information about the angles of the ultrasound probe 2) and the reflected-wave data received at each of the angles.

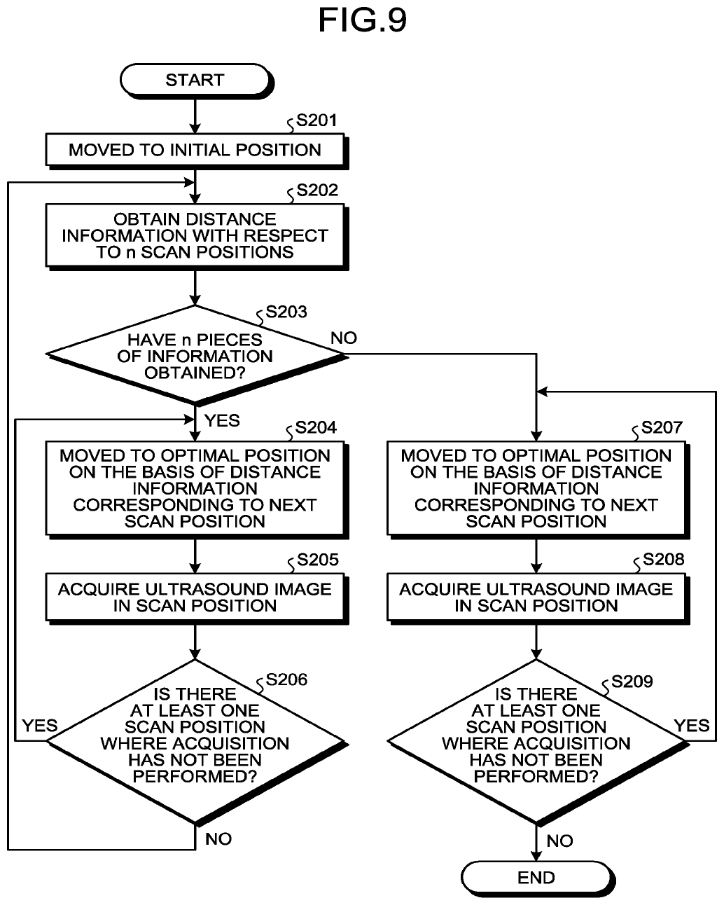

Next, a process performed by the ultrasound diagnosis apparatus 1 according to the second embodiment will be explained, with reference to FIG. 9. FIG. 9 is a flowchart for explaining a procedure in the process performed by the ultrasound diagnosis apparatus 1 according to the second embodiment. In the present example, steps S201, S203, S204, S206, S207, and S209 in FIG. 9 are steps executed as a result of the processing circuitry 55 reading the program corresponding to the mechanism controlling function 554 from the memory 54. Step S202 is a step executed as a result of the processing circuitry 55 reading the programs corresponding to the controlling function 551 and the detecting function 553 from the memory 54. Steps S205 and S208 are steps executed as a result of the processing circuitry 55 reading the program corresponding to the controlling function 551 from the memory 54.

In the ultrasound diagnosis apparatus 1 according to the first embodiment, the processing circuitry 55 moves the ultrasound probe 2 to the initial position (step S201). Further, the processing circuitry 55 exercises control so that pieces of distance information are obtained with respect to n scan positions (step S202) and judges whether or not the number of obtained pieces of distance information is equal to n (step S203).

In this situation, the number of scan positions related to the obtainment of the distance information acquired in a deflection scan in one session is determined on the basis of a deflectable angle and the scan interval. Further, the judgement as to whether or not the number of obtained pieces of distance information is equal to n may be made, for example, on the basis of distance information detected by the detecting function 553. In that situation, for example, the processing circuitry 55 may mutually compare the pieces of distance information detected with respect to the scan positions to determine, when the difference between pieces of distance information detected in adjacent scan positions is equal to or larger than a threshold value, that the latter of the scan positions corresponding to the two pieces of distance information exhibiting the difference equal to or larger than the threshold value is positioned outside the scan target site and thus determine that the number of obtained pieces of distance information is not equal to n, since the obtained piece of distance information is not included in the count.

In one example, when the difference between the distance information with respect to the scan portion P2 and the distance information with respect to the scan position P3 is equal to or larger than the threshold value, the processing circuitry 55 does not include the piece of distance information corresponding to the scan position P3 in the count.

In the judging process at step S203, when it is determined that n pieces of distance information have been obtained (step S203: Yes), the processing circuitry 55 moves the ultrasound probe 2 to the optimal position (the probe position) on the basis of the distance information corresponding to the next scan position (step S204). Subsequently, the processing circuitry 55 acquires an ultrasound image in the scan position (step S205) and judges whether or not there is at least one scan position in which acquisition has not yet been performed (step S206).

In this situation, when there is at least one scan position in which acquisition has not yet been performed (step S206: Yes), the processing circuitry 55 returns to step S204 and continues the process. On the contrary, when there is no scan position in which acquisition has not yet been performed (step S206: No), the processing circuitry 55 returns to step S202 and continues the process.

In the judgment at step S203, when the number of obtained pieces of distance information is not equal to n (step S203: No), the processing circuitry 55 moves the ultrasound probe 2 to the optimal position (the probe position) on the basis of the distance information corresponding to the next scan position (step S207). After that, the processing circuitry 55 acquires an ultrasound image in the scan position (step S208) and judges whether or not there is at least one scan position in which acquisition has not yet been performed (step S209).

When there is at least one scan position in which acquisition has not yet been performed (step S209: Yes), the processing circuitry 55 returns to step S207 and continues the process. On the contrary, when there is no scan position in which acquisition has not yet been performed (step S209: No), the processing circuitry 55 ends the process.

As explained above, according to the second embodiment, the detecting function 553 is configured to detect the distance information between the body surface of the subject and the ultrasound wave transmission-reception surface of the ultrasound probe 2, with respect to each of the plurality of scan positions set along the moving direction of the ultrasound probe 2. Every time the distance information is detected by the detecting function 553, the mechanism controlling function 554 is configured to sequentially move the ultrasound probe 2 to the probe position corresponding to the scan position, based on the distance information with respect to the scan position.

In other words, in the first scan position, the detecting function 553 is configured to detect the distance information between the body surface of the subject and the ultrasound wave transmission-reception surface of the ultrasound probe 2 with respect to the third scan position set along the body surface of the subject. After the ultrasound scan in the first scan position is performed, the mechanism controlling function 554 is configured to move the ultrasound probe 2 to the third scan position and to also move the ultrasound probe 2 so that the distance between the body surface of the subject and the ultrasound wave transmission-reception surface with respect to the third scan position is equal to the distance based on the distance information corresponding to the third scan position. The controlling function 551 is configured to control the ultrasound scan performed in the third scan position. Consequently, the ultrasound diagnosis apparatus 1 according to the second embodiment is able to further shorten the time period required by the automatic scan and makes it possible to improve efficiency of the scans using the robot.

Further, according to the second embodiment, the controlling function 551 is configured to scan the body surface in the positions from the first scan position to the third scan position, by performing the deflection scan in which the ultrasound wave transmission and reception direction is varied. On the basis of the ultrasound wave obtained in the deflection scan, the detecting function 553 is configured to detect the distance information between the body surface of the subject and the ultrasound wave transmission-reception surface of the ultrasound probe with respect to the third scan position. Consequently, the ultrasound diagnosis apparatus 1 according to the second embodiment makes it possible to easily detect the pieces of distance information simultaneously with respect to the plurality of scan positions.

### Third Embodiment

The first and the second embodiments have thus been explained. It is, however, possible to carry out the present disclosure in various different modes other than those described in the first and the second embodiments.

In the first and the second embodiments, the example was explained in which the single ultrasound probe (i.e., the ultrasound probe 2) performs the distance measuring process and the ultrasound scan; however, possible embodiments are not limited to this example. For instance, it is acceptable to provide an ultrasound probe used for the distance measuring process and another ultrasound probe used for the ultrasound scan.

In that situation, the ultrasound probe 2 according to the third embodiment includes a first ultrasound probe and a second ultrasound probe. The detecting function 553 according to the third embodiment is configured to detect distance information between the body surface of the subject and the ultrasound wave transmission-reception surface of the ultrasound probe, on the basis of an ultrasound wave transmitted and received by the first ultrasound probe. The controlling function 551 according to the third embodiment is configured to control the ultrasound scan performed on the subject, by controlling ultrasound wave transmission and reception by the second ultrasound probe.

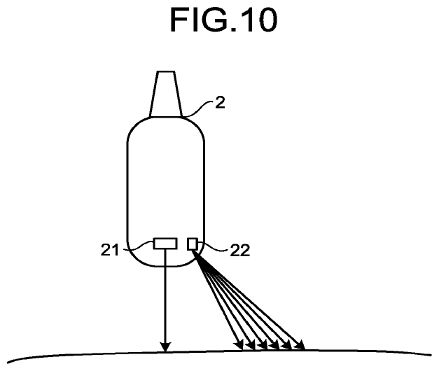

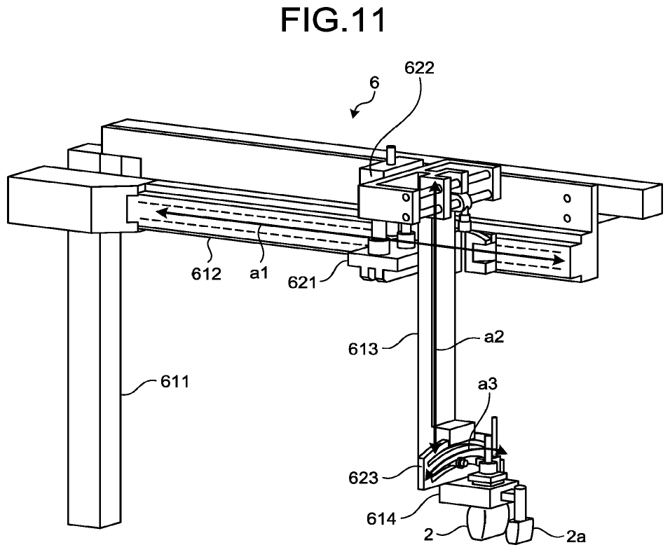

In this situation, the first ultrasound probe and the second ultrasound probe may be contained in mutually the same casing or may be contained in mutually-different casings. Next, examples of the ultrasound probe according to the third embodiment will be explained, with reference to FIGS. 10 and 11. FIGS. 10 and 11 are drawings for explaining the examples of the ultrasound probe according to the third embodiment. FIG. 10 illustrates an example in which the first ultrasound probe and the second ultrasound probe are contained in mutually the same casing. FIG. 11 illustrates an example in which the first ultrasound probe and the second ultrasound probe are contained in mutually-different casings.

As illustrated in FIG. 10, the ultrasound probe 2 according to the third embodiment has, inside the casing, a group of piezoelectric transducer elements 21 and another group of piezoelectric transducer elements 22. The piezoelectric transducer elements are each provided with a matching layer, a backing member, and the like. Further, each of the piezoelectric transducer elements is configured to generate an ultrasound wave on the basis of a drive signal supplied thereto from the transmission and reception circuitry 51.

For example, on the basis of a drive signal supplied thereto from the transmission and reception circuitry 51 under control of the controlling function 551, the group of piezoelectric transducer elements 21 (the second ultrasound probe) performs an ultrasound scan to acquire biological information, by transmitting and receiving an ultrasound wave to and from the subject. Further, for example, on the basis of a drive signal supplied thereto from the transmission and reception circuitry 51 under control of the controlling function 551, the piezoelectric transducer elements 22 (the first ultrasound probe) perform an ultrasound scan to acquire distance information, by transmitting and receiving an ultrasound wave to and from the subject.

An example of the process performed by the ultrasound probe 2 according to the third embodiment will be explained, with reference to FIG. 5. For example, when the ultrasound probe 2 reaches the scan position P1, the controlling function 551 controls the transmission and reception circuitry 51 so that an ultrasound wave is transmitted from the piezoelectric transducer elements 22 in the ultrasound probe 2, and a reflected wave is received. Further, the image generating function 552 generates ultrasound image data on the basis of the reflected-wave data generated by the transmission and reception circuitry 51. The detecting function 553 calculates the distance between the body surface in the scan target site and the ultrasound wave transmission-reception surface of the ultrasound probe 2, by using the ultrasound image data received from the image generating function 552.

When the distance between the ultrasound probe 2 and the body surface has been detected, the mechanism controlling function 554 controls the mechanical mechanism 6 so as to move the ultrasound probe 2 to a probe position derived on the basis of the detected distance information.

When the ultrasound probe 2 has been moved to the probe position, the controlling function 551 controls the transmission and reception circuitry 51 so that an ultrasound wave is transmitted from the piezoelectric transducer elements 21 in the ultrasound probe 2, and a reflected wave is received. Further, the image generating function 552 generates ultrasound image data on the basis of the reflected-wave data generated by the transmission and reception circuitry 51. The process described above is performed in each of the scan positions set along the body surface of the subject.

In another example where the first ultrasound probe and the second ultrasound probe are contained in mutually-different casings, for example, as illustrated in FIG. 11, the ultrasound diagnosis apparatus 1 according to the third embodiment includes the ultrasound probe 2 (the second ultrasound probe) and an ultrasound probe 2a (the first ultrasound probe). Each of the ultrasound probes is configured to generate an ultrasound wave on the basis of a drive signal supplied thereto from the transmission and reception circuitry 51.

For example, the ultrasound probe 2 is configured to perform an ultrasound scan to acquire biological information, by transmitting and receiving an ultrasound wave to and from the subject, on the basis of a drive signal supplied thereto from the transmission and reception circuitry 51 under the control of the controlling function 551. Further, for example, the ultrasound probe 2a is configured to perform an ultrasound scan to acquire distance information by transmitting and receiving an ultrasound wave to and from the subject, on the basis of a drive signal supplied thereto from the transmission and reception circuitry 51 under the control of the controlling function 551.

An example of the process performed by the ultrasound probe 2 and the ultrasound probe 2a according to the third embodiment will be explained, with reference to FIG. 5. For example, when the ultrasound probe 2a reaches the scan position P1, the controlling function 551 controls the transmission and reception circuitry 51 so that an ultrasound wave is transmitted from the ultrasound probe 2a, and a reflected wave is received. Further, the image generating function 552 generates ultrasound image data on the basis of the reflected-wave data generated by the transmission and reception circuitry 51. The detecting function 553 calculates the distance between the body surface in the scan target site and the ultrasound wave transmission-reception surface of the ultrasound probe 2a, by using the ultrasound image data received from the image generating function 552.

When the distance between the ultrasound probe 2a and the body surface has been detected, the mechanism controlling function 554 controls the mechanical mechanism 6 so as to move the ultrasound probe 2 to a probe position derived on the basis of the detected distance information. In this situation, the mechanism controlling function 554 derives the probe position while taking into consideration the difference between the positions of the ultrasound wave transmission-reception surfaces, when the ultrasound probe 2 and the ultrasound probe 2a are held by the mechanical mechanism 6. In other words, while the ultrasound probe 2 and the ultrasound probe 2a are held by the mechanical mechanism 6, when the positions of the ultrasound wave transmission-reception surfaces in the vertical direction are the same as each other, the mechanism controlling function 554 derives the probe position from the detected distance information on the assumption that there is no difference between the positions of the ultrasound wave transmission-reception surfaces of the ultrasound probe 2 and the ultrasound probe 2a.

On the contrary, while the ultrasound probe 2 and the ultrasound probe 2a are held by the mechanical mechanism 6, when the positions of the ultrasound wave transmission-reception surfaces in the vertical direction are different from each other, the mechanism controlling function 554 derives a probe position from the difference between the positions of the ultrasound wave transmission-reception surfaces in the vertical direction and the detected distance information. For example, the mechanism controlling function 554 adds, as offset information, the difference between the positions of the ultrasound wave transmission-reception surfaces in the vertical direction to the detected distance information and further derives the probe position on the basis of the distance information to which the offset information has been added.

When the ultrasound probe 2 has been moved to the probe position corresponding to the scan position P1, the controlling function 551 controls the transmission and reception circuitry 51 so that an ultrasound wave is transmitted from the ultrasound probe 2, and a reflected wave is received. Further, the image generating function 552 generates ultrasound image data on the basis of the reflected-wave data generated by the transmission and reception circuitry 51. The process described above is performed in each of the scan positions set along the body surface of the subject.

In the first and the second embodiments described above, the example was explained in which the automatic scan is performed by using only the first mechanism unit 621 and the second mechanism unit 622 in the mechanical mechanism 6; however, possible embodiments are not limited to this example. For instance, the third mechanism unit 623 may be used.

Further, in the embodiments described above, the example was explained in which the ultrasound probe 2 is connected to the apparatus main body 5 via a cable; however, possible embodiments are not limited to this example. For instance, the ultrasound wave transmission and reception by the ultrasound probe may be controlled wirelessly. In that situation, for example, a transmission and reception circuitry is built in the probe main body of the ultrasound probe, so that the ultrasound wave transmission and reception by the ultrasound probe is wirelessly controlled by another apparatus. The ultrasound diagnosis apparatus according to the present embodiments may be configured so as to include only one or more wireless ultrasound probes configured in this manner.

In the embodiments described above, the examples were explained in which the ultrasound diagnosis apparatus 1 performs the various types of processes; however, possible embodiments are not limited to those examples. For instance, one or more of the processes described above as being performed by the ultrasound diagnosis apparatus 1 may be performed by the mechanical mechanism 6. Further, for example, all of the processes described above as being performed by the ultrasound diagnosis apparatus 1 may be performed by the mechanical mechanism 6 serving as an ultrasound scanning support apparatus.

The term “processor” used in the above explanations denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). The processors realize the functions by reading and executing the programs saved in the storage circuit. In this situation, instead of saving the programs in the storage circuit, it is also acceptable to directly incorporate the programs in the circuits of the processors. In that situation, the processors realize the functions by reading and executing the programs incorporated in the circuits thereof. Further, the processors in the present embodiments do not each necessarily have to be structured as a single circuit. It is also acceptable to structure one processor by combining together a plurality of independent circuits so as to realize the functions thereof.

Further, the constituent elements of the apparatuses and the devices presented in the drawings for explaining the embodiments are based on functional concepts. Thus, it is not necessary to physically configure the constituent elements as indicated in the drawings. In other words, specific modes of distribution and integration of the apparatuses and the devices are not limited to those illustrated in the drawings. It is acceptable to functionally or physically distribute or integrate all or a part of the apparatuses and the devices in any arbitrary units, depending on various loads and the status of use. Further, all or an arbitrary part of the processing functions performed by the apparatuses and the devices may be realized by a CPU and a program analyzed and executed by the CPU or may be realized as hardware using wired logic.

Further, it is possible to realize the processing methods described in the present embodiments, by causing a computer such as a personal computer or a workstation to execute a processing program prepared in advance. It is possible to distribute the processing program via a network such as the Internet. Further, it is also possible to record the processing program onto a non-transitory computer-readable recording medium such as a hard disk, a flexible disk (FD), a Compact Disk Read-Only Memory (CD-ROM), a Magneto-Optical (MO) disk, a Digital Versatile Disk (DVD), a flash memory such as a Universal Serial Bus (USB) memory or a Secure Digital (SD) card memory, or the like, so that the processing program is executed as being read from the non-transitory recording medium by a computer.

As explained above, according to at least one aspect of the present embodiments, it is possible to improve efficiency of the scans using the robot.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound automatic scanning system comprising:
at least one ultrasound probe configured to transmit and receive an ultrasound wave;
a mechanical mechanism configured to hold the ultrasound probe and to move the ultrasound probe while an ultrasound wave transmission-reception surface of the ultrasound probe is directed toward a subject; and
processing circuitry configured to
detect, on a basis of the ultrasound wave transmitted and received by the ultrasound probe, distance information between a body surface of the subject and the ultrasound wave transmission-reception surface of the ultrasound probe, with respect to a first scan position and a second scan position set along the body surface of the subject,
control the mechanical mechanism so as to move the ultrasound probe on a basis of the distance information, and
control ultrasound scans performed in the first scan position and in the second scan position on the subject by the ultrasound probe moved by the mechanical mechanism, wherein
the processing circuitry is further configured to control the mechanical mechanism so as to move the ultrasound probe to the second scan position after the distance information detection and the ultrasound scan in the first scan position are performed.

2. The ultrasound automatic scanning system according to claim 1, wherein the processing circuitry is configured to
detect, on a basis of an ultrasound wave transmitted and received after a move to a scan position, distance information between the body surface of the subject and the ultrasound wave transmission-reception surface of the ultrasound probe with respect to the scan position,
move the ultrasound probe so that a distance between the body surface of the subject and the ultrasound wave transmission-reception surface with respect to the scan position is equal to a distance based on the distance information, and
control the ultrasound scan performed in the scan position on the subject by the post-move ultrasound probe.

3. The ultrasound automatic scanning system according to claim 1, wherein the processing circuitry is configured to
detect, in the first scan position, distance information between the body surface of the subject and the ultrasound wave transmission-reception surface of the ultrasound probe with respect to a third scan position set along the body surface of the subject,
move, after the ultrasound scan in the first scan is performed, the ultrasound probe to the third scan position and also move the ultrasound probe so that a distance between the body surface of the subject and the ultrasound wave transmission-reception surface with respect to the third scan position is equal to a distance based on the distance information corresponding to the third scan position, and
control an ultrasound scan performed in the third scan position.

4. The ultrasound automatic scanning system according to claim 3, wherein the processing circuitry is configured to
cause the body surface to be scanned in positions from the first scan position to the third scan position by performing a deflection scan in which an ultrasound wave transmission and reception direction is varied, and
detect the distance information between the body surface of the subject and the ultrasound wave transmission-reception surface of the ultrasound probe with respect to the third scan position, on a basis of an ultrasound wave obtained in the deflection scan.

5. The ultrasound automatic scanning system according to claim 1, wherein a position of the ultrasound probe with respect to the body surface of the subject is derived from the distance information so that a focal point of the ultrasound wave falls within a region of interest.

6. The ultrasound automatic scanning system according to claim 1, wherein
the ultrasound probe includes a first ultrasound probe and a second ultrasound probe,
the processing circuitry is configured to
detect the distance information between the body surface of the subject and the ultrasound wave transmission-reception surface of the ultrasound probe, on a basis of an ultrasound wave transmitted and received by the first ultrasound probe, and
control the ultrasound scan performed on the subject, by controlling ultrasound wave transmission and reception performed by the second ultrasound probe.

7. The ultrasound automatic scanning system according to claim 6, wherein the first ultrasound probe and the second ultrasound probe are contained in a mutually same casing and are held by the mechanical mechanism.

8. The ultrasound automatic scanning system according to claim 6, wherein the first ultrasound probe and the second ultrasound probe are contained in mutually-different casings and are each held by the mechanical mechanism.

9. The ultrasound automatic scanning system according to claim 1, wherein interval distances between a plurality of scan positions set along the body surface of the subject are each equal to or shorter than 2 mm.

10. An ultrasound diagnosis apparatus comprising:
at least one ultrasound probe configured to transmit and receive an ultrasound wave; and
processing circuitry configured to
detect, on a basis of the ultrasound wave transmitted and received by the ultrasound probe, distance information between a body surface of a subject and an ultrasound wave transmission-reception surface of the ultrasound probe, with respect to a first scan position and a second scan position set along the body surface of the subject, the ultrasound probe being moved by a mechanical mechanism configured to hold the ultrasound probe and to move the ultrasound probe while the ultrasound wave transmission-reception surface of the ultrasound probe is directed toward the subject,
control the mechanical mechanism so as to move the ultrasound probe on a basis of the distance information, and
control ultrasound scans performed in the first scan position and in the second scan position on the subject by the ultrasound probe moved by the mechanical mechanism, wherein
the processing circuitry is further configured to control the mechanical mechanism so as to move the ultrasound probe to the second scan position after the distance information detection and the ultrasound scan in the first scan position are performed.

11. An ultrasound scanning support apparatus comprising:
a mechanical mechanism configured to hold at least one ultrasound probe that transmits and receives an ultrasound wave and to move the ultrasound probe while an ultrasound wave transmission-reception surface of the ultrasound probe is directed toward a subject; and
processing circuitry configured to
detect, on a basis of the ultrasound wave transmitted and received by the ultrasound probe moved by the mechanical mechanism, distance information between a body surface of the subject and the ultrasound wave transmission-reception surface of the ultrasound probe, with respect to a first scan position and a second scan position set along the body surface of the subject, and
control the mechanical mechanism so as to move the ultrasound probe on a basis of the distance information, wherein
the processing circuitry is further configured to control the mechanical mechanism so as to move the ultrasound probe to the second scan position after the distance information detection and an ultrasound scan in the first scan position are performed.
